(19) United States
(12) Patent Application Publication
(10) Pub. No.: US 2021/0015699 A1
(43) Pub. Date: Jan. 21, 2021

(54) GLOVE MASSAGER

(71) Applicant: Jianhan ZOU, Fujian (CN)

(72) Inventors: Jianhan ZOU, Fujian (CN); Guoyun TANG, Fujian (CN); Weijie CHEN, Fujian (CN)

(73) Assignee: Jianhan ZOU, Fujian (CN)

(21) Appl. No.: 16/628,576

(22) PCT Filed: Sep. 27, 2018
(86) PCT No.: PCT/CN2018/108052

§ 371 Date: Jun. 10, 2020

(30) Foreign Application Priority Data
Sep. 28, 2017 (CN) 201721258531.6

(51) Int. Cl. A61H 9/00

(52) U.S. Cl. CPC A61H 9/0092; A61H 2205/067; A61H 2201/1638; A61H 2201/5007; A61H 2201/1409; A61H 2201/0207; A61H 2201/1645

(57) ABSTRACT

Provided is a glove massager, comprising an air bag assembly (2) and an air pump (1), wherein the air bag assembly (2) comprises a first massaging portion and a second massaging portion (21); the second massaging portion (21) is provided with an annular air bag for fingers to pass through same; and the air pump (1) is connected to the annular air bag, and the annular air bag can massage the fingers of a user. The massager can massage the palm and the fingers at the same time and has various massage functions and an excellent massage effect. The usage of the annular air bag can realize omnidirectional massage for the fingers, and can also improve the massage effect for the fingers.

### TECHNICAL FIELD

The invention relates to a massager, and in particular to a glove massager for massaging the hands of users.

### DESCRIPTION OF RELATED ART

The traditional glove massager includes an air bag assembly and an air pump, wherein the air pump is communicated with the air bag assembly to drive the air bag assembly to expand and contract to massage the hands of users. However, the traditional glove massager can only massage the palms of users, thereby having only one function and being low in comfort degree, such as Chinese Utility Model Patent Application No. 200820171506.9.

### BRIEF SUMMARY OF THE INVENTION

### Technical Issue

The invention provides a glove massager which overcomes the defect of the single massage function in the prior art.

### Solution to the Technical Issue

The technical solution adopted by the invention to settle the technical issue is as follows:

A glove massager includes an air bag assembly (2) and an air pump (1), wherein the air bag assembly (2) includes second massage parts (21) provided with ring-shaped air bags permitting fingers to penetrate through, and the air pump (1) is connected with the ring-shaped air bags which are used to massage the fingers of a user.

### Beneficial Effects of the Invention

Compared with the prior art, this technical solution has the following advantages:

A first massage part has a front part and a back part provided with first air bags used to massage the palm of a user, and the second massage parts are provided with the ring-shaped air bags permitting fingers to penetrate through so as to massage the fingers of the user, so that at least the palm and the fingers can be massaged at the same time, a massage function is diversified, and the massage effect is good; and besides, the ring-shaped air bags are adopted to realize all-around massage on the fingers, so that the massage effect on the fingers can be improved.

The first massage part is folded to at least form the front part and the back part, and the second massage parts are connected with the front part or the back part, so that the structure is simple, the manufacturing cost is low, and assembling is convenient.

The first air bags are arranged at the front part and the back part and correspond to the center of the palm to massage the center of the palm of the user.

Two second air bags are also arranged at the front part and the back part and are distributed on the left side and the right side of each first air bag to massage the left part and the right part of the palm.

The number of the second massage parts which are distributed in parallel is at least four, so that the glove massager can be worn by the user conveniently.

Third air bags are located between the second massage parts and the first air bags and correspond to the roots of fingers to massage the roots of the fingers.

Heating sheets are arranged at the front part and/or the back part of the first massage part so as to fulfill massage through the air bags and also through heating, and thus, the massage effect is good.

Silica gel massage sheets are arranged at the front part and/or the back part of the first massage part so as to further improve the massage effect.

The air bag assembly is arranged in a through space of a shell part, so that the structure is simple, and massaging by the user is facilitated.

The shell part also includes a shell including an upper cover and a lower cover, wherein a body is fixedly arranged between the upper cover and the lower cover which are fixedly connected together; the air pump is fixedly connected into the shell, and air valves, a circuit board, and an operation part are connected with the shell part, so that assembling is convenient, and the structure is simple and compact; and the air pump is connected with the first air bags and the ring-shaped air bags through the air valves, so that the user can conveniently select a massage position as required.

### BRIEF DESCRIPTION OF THE SEVERAL VIEWS OF THE DRAWINGS

FIG. 1 is one perspective view of a glove massager in a specific implementation;

FIG. 2 is another perspective view of the glove massage in the specific implementation;

FIG. 3 is a perspective exploded view of the glove massage in the specific implementation;

FIG. 4 is one perspective exploded view of an air bag assembly of the glove massage in the specific implementation;

FIG. 5 is another perspective exploded view of the air bag assembly of the glove massage in the specific implementation.

### DETAILED DESCRIPTION OF THE INVENTION

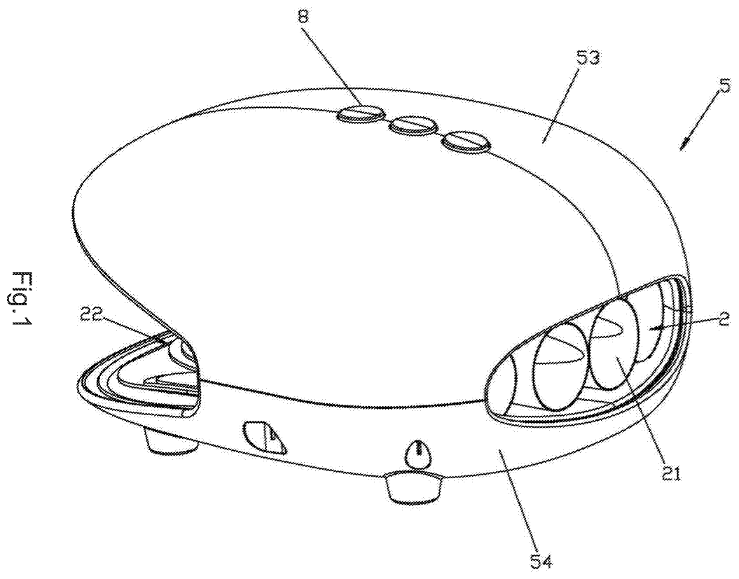

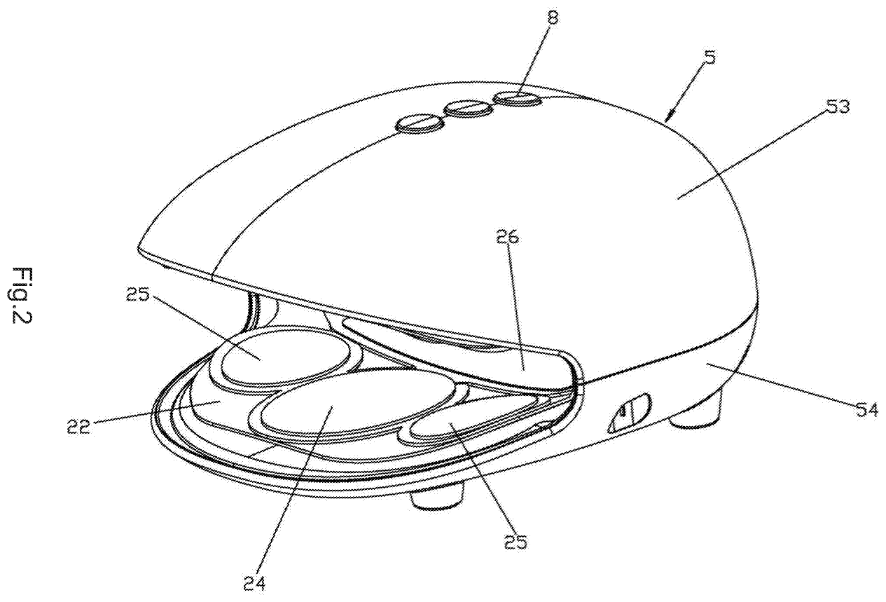

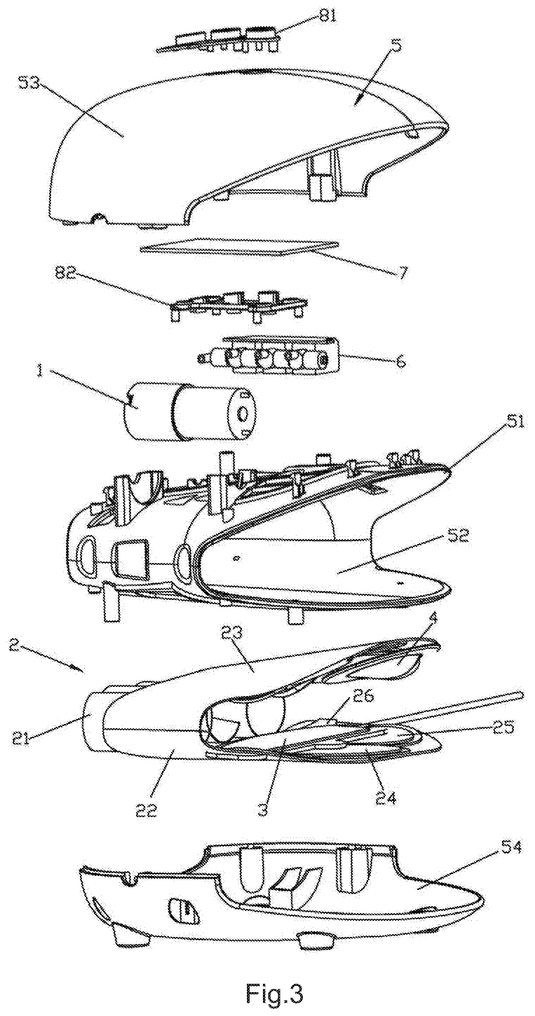

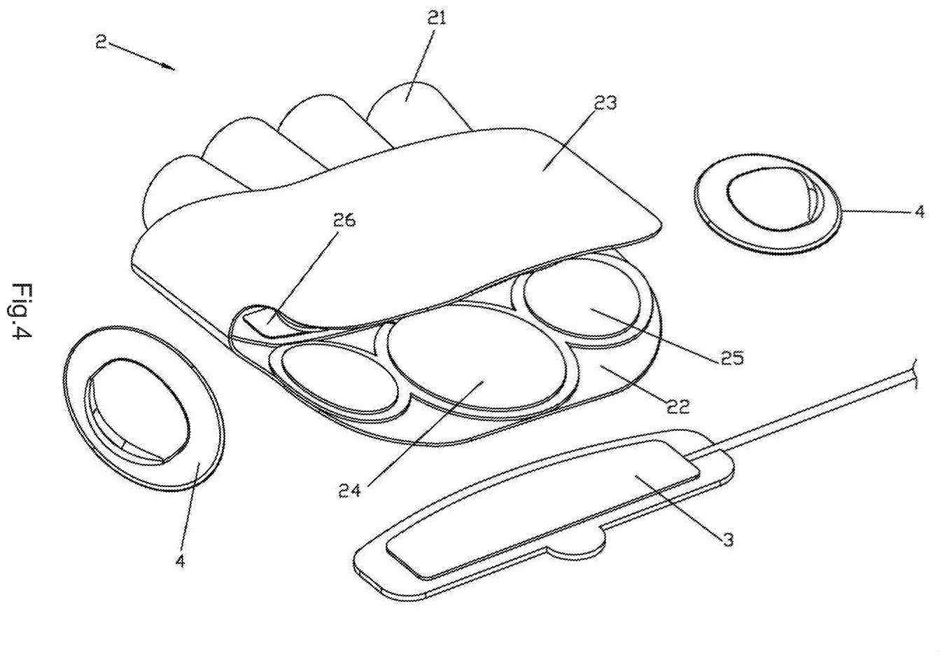

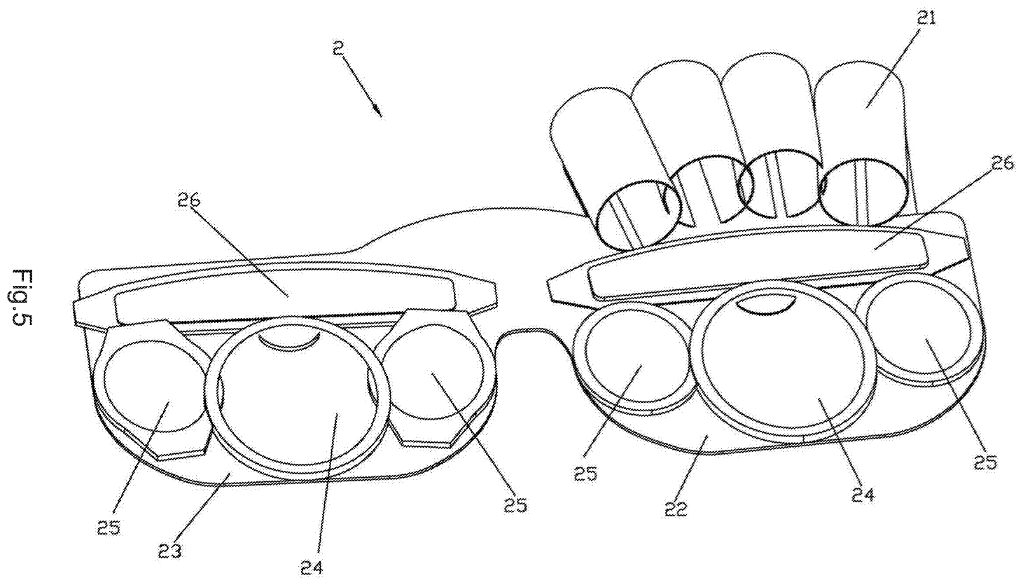

Referring to FIG. 1 to FIG. 5, a glove massager including an air bag assembly 2, an air pump 1, heating sheets 3, massage sheets 4, a shell part 5, air valves 6, a circuit board 7 and an operation part 8 to be operated by a user. The air bag assembly 2 includes a first massage part and second massage parts 21.

The first massage part has at least a front part 22 and a back part 23 which respectively correspond to the front and the back of a palm. In this specific implementation, the first massage part is folded to at least form the front part 22 and the back part 23. The front part 22 and the back part 23 are provided with first air bags 24 which are arranged at positions, corresponding to the center of a palm, of the front part 22 and the back part 23; as required, the front part 22 and the back part 23 are each provided with two second air bags 25 distributed on the left side and the right side of each first air bag 24; and as required, the front part 22 and the back part 23 are provided with third air bags 26 which are located between the second massage parts 21 and the first air bags 24 and correspond to the roots of fingers.

The second massage parts 21 are provided with the ring-shaped air bags permitting fingers to penetrate through, and the number of the second massage parts 21 which are distributed in parallel is four; and besides, the second massage parts 21 are connected with the front part 22 or the back part 23, for example, the second massage parts 21 are connected with the front part 22, or the second massage parts 21 are connected with the back part 23.

The air pump 1 can be communicated with the first air bags 24, the second air bags 25, the third air bags 26 and the ring-shaped air bags. As required, the air pump 1 is connected with the first air bags 24, the second air bags 25, the third air bags 26 and the ring-shaped air bags through the air valves 6.

The heating sheets 3 are arranged at the front part 22 and/or the back part 23 of the first massage part. The massage sheets 4, such as silica gel massage sheets, are arranged at the front part 22 and/or the back part 23 of the first massage part.

In this specific implementation, the shell part 5 includes a body 51 and a shell, wherein the body 51 has a through space 52, and the air bag assembly 2 is arranged in the through space 52 of the shell part 5 in a connected manner; the shell includes an upper cover 53 and a lower cover 54; and the body 51 is fixedly arranged between the upper cover 53 and the lower cover 54 which are fixedly connected together. The air pump 1, the air valves 6 and the circuit board 7 are fixedly connected into the shell, and the operation part 8, such as keys or a key panel, is arranged on the upper cover 53 of the shell part 5. The operation part 8 is arranged on the shell part 5 and electrically connected with the circuit board 7 which is electrically connected with the air pump 1 and the air valves 6. The operation part 8 includes keys 81 and a key fixing plate 82.

### A LIST OF REFERENCE DOCUMENTS

### Patent Documents

Patent document 1: Chinese Utility Model Patent Application No. 200820171506.9.

1-11. (canceled)

12. A glove massager, comprising an air bag assembly, an air pump, air valves, a circuit board and a shell part, wherein the air bag assembly, the air pump, the air valves and the circuit board are arranged in the shell part; the circuit board is electrically connected with the air pump and the air valves; the air bag assembly comprises second massage parts provided with ring-shaped air bags permitting fingers to penetrate through; and the air pump is connected with the ring-shaped air bags through the air valves, and the ring-shaped air bags are used to massage fingers of a user.

13. The glove massager according to claim 1, also comprising a first massage part at least having a front part which corresponds to a palm and is provided with a first air bag, and the air pump is connected with the first air bag through the corresponding air valve.

14. The glove massager according to claim 1, wherein the shell part comprises a body having a through space, the air bag assembly is arranged in the through space in a connected manner, and the body is fixedly arranged in the shell part.

15. The glove massager according to claim 2, wherein the first massage part also has a back part corresponding a back of a hand, and the first massage part is folded to from the front part and the back part; and the second massage parts are connected with the front part or the back part.

16. The glove massager according to claim 2, wherein the first air bag is arranged at a position, corresponding to a center of the palm, of the front part to massage the center of the palm of the user.

17. The glove massager according to claim 5, wherein two second air bags are also arranged at the front part and are distributed on a left side and a right side of the first air bag, and the air pump is connected with the second air bags through the air valves.

18. The glove massager according to claim 3, wherein the shell part also comprises a shell including an upper cover and a lower cover, and the body is fixedly arranged between the upper cover and the lower cover.

19. The glove massager according to claim 1, wherein the number of the second massage parts which are distributed in parallel is at least four.

20. The glove massager according to claim 2, wherein a third air bag is also arranged at the front part, is located between the second massage parts and the first air bag and corresponds to roots of fingers to massage the roots of the fingers.

21. The glove massager according to claim 2, also comprising a heating sheet arranged at the front part of the first massage part.

22. The glove massager according to claim 1, also comprising an operation part which is to be operated by the user, arranged in the shell part and electrically connected with the circuit board.
